(19) United States
(12) Patent Application Publication
(10) Pub. No.: US 2024/0047740 A1
(43) Pub. Date: Feb. 8, 2024

(54) IONIC ORGANIC FRAMEWORK FOR ALL-SOLID SECONDARY BATTERY, ELECTROLYTE CONTAINING SAME, AND ALL-SOLID SECONDARY BATTERY INCLUDING SAME

(71) Applicant: INDUSTRY-UNIVERSITY COOPERATION FOUNDATION HANYANG UNIVERSITY ERICA CAMPUS, Gyeonggi-do (KR)

(72) Inventors: Jong-Ho Kim, Gyeonggi-do (KR); Jung Hyun Park, Gyeonggi-do (KR); Tae Woog Kang, Gyeonggi-do (KR); Jong-Min Ju, Gyeonggi-do (KR)

(21) Appl. No.: 18/266,476

(22) PCT Filed: Mar. 8, 2022
(86) PCT No.: PCT/KR2022/003248

§ 371 Date: Oct. 20, 2023

(30) Foreign Application Priority Data
Mar. 18, 2021 (KR) 10-2021-0035203
Feb. 14, 2022 (KR) 10-2022-0019028

(51) Int. Cl. H01M 10/0565; H01M 10/052

(52) U.S. Cl. CPC H01M 10/0565; H01M 10/052; H01M 2300/0082

(57) ABSTRACT

Provided is an ionic organic framework electrolyte for an all-solid secondary battery, more particularly to an ionic organic framework for a solid electrolyte of an all-solid secondary battery, the solid electrolyte having effectively controlled structure and characteristics, and excellent lithium-ion conductivity and stability, an electrolyte containing the same, and an all-solid secondary battery including the same.

### TECHNICAL FIELD

The present disclosure relates to an ionic organic framework electrolyte for an all-solid secondary battery, more particularly to an ionic organic framework for a solid electrolyte of an all-solid secondary battery, the solid electrolyte having effectively controlled structure and characteristics, and excellent lithium-ion conductivity and stability, an electrolyte containing the same, and an all-solid secondary battery including the same.

### BACKGROUND ART

The solid electrolyte for a secondary battery is the core technology of the all-solid secondary battery which is drawing a lot of interests recently in the field of electric vehicles and future energy materials. As the electrolyte of the all-solid secondary battery, organic electrolytes (dry polymer electrolytes), inorganic electrolytes (sulfides, etc.), composite electrolytes (nanoparticle fillers and polymers), etc. are being researched. For example, Korean Patent Publication No. 10-2019-0033422 discloses a polymer electrolyte using polymers such as poly(ethylene oxide) (PEO). However, due to the use of polymers, the preparation process is complicated. Further, it is difficult to effectively control the desired ionic property, and stability is decreased.

Accordingly, development of a new carbon material-based solid electrolyte which has crystallinity without using a polymer and ionic conductivity of which is controllable by freely selecting an anion is necessary.

### DISCLOSURE OF THE INVENTION

### Technical Problem

The present disclosure is directed to providing a new solid electrolyte material which has crystallinity without using a polymer and ionic conductivity of which is controllable by freely selecting an anion, as an electrolyte for an all-solid secondary battery, an electrolyte containing the same, and an all-solid secondary battery including the same.

However, the technical problem to be solved by the present disclosure is not limited to that described above and other unmentioned problems will be clearly understood by those having ordinary skill in the art from the following description.

### Technical Solution

The present disclosure provides an ionic organic framework for an all-solid secondary battery, wherein the organic framework contains a compound capable of forming a cation through protonation in the framework, a counteranion for a secondary battery is bonded to the cation, and the organic framework has crystallinity.

In an exemplary embodiment of the present disclosure, the organic framework is formed from condensation between an aromatic compound having at least three functional groups that can participate in condensation and another aromatic compound having two or more functional groups that can participate in condensation.

In an exemplary embodiment of the present disclosure, the aromatic compound is triazine and the another aromatic compound is at least one of pyridine or imidazole.

In an exemplary embodiment of the present disclosure, the ionic organic framework for an all-solid secondary battery has a structure of Chemical Formula (1) or Chemical Formula (2).

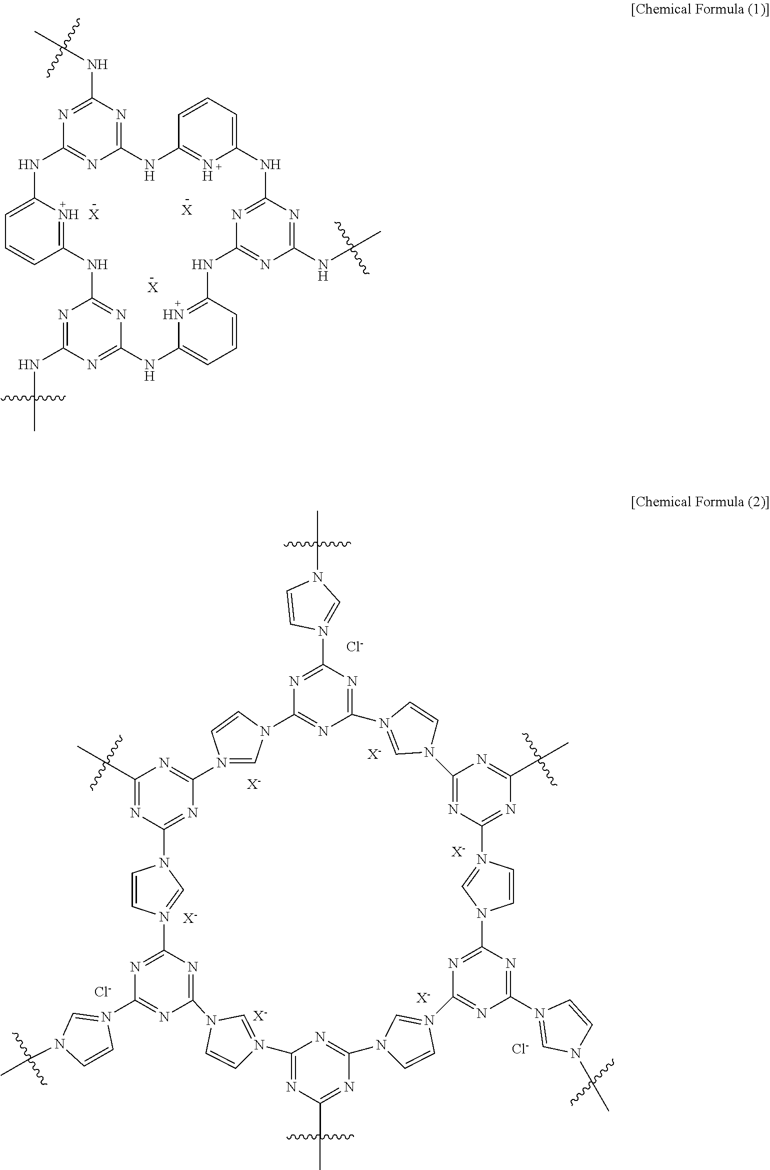

In the Chemical Formula (1) and (2), X is a counterion of a lithium battery.

In an exemplary embodiment of the present disclosure, the organic framework is for an electrolyte of an all-solid secondary battery.

In an exemplary embodiment of the present disclosure, the organic framework of Formula (1) is obtained by reacting cyanuric chloride with 2,6-diaminopyridine and has a structure wherein the triazine of the cyanuric chloride is connected to the pyridine of the 2,6-diaminopyridine by an amine group.

In an exemplary embodiment of the present disclosure, the organic framework of Formula (2) is obtained by reacting cyanuric chloride with imidazole and has a structure wherein the triazine of the cyanuric chloride is bonded to the imidazole.

The present disclosure also provides an electrolyte containing the ionic organic framework for an all-solid secondary battery.

The present disclosure also provides an all-solid secondary battery including the electrolyte.

The present disclosure also provides a method for preparing an ionic organic framework for an all-solid secondary battery, which includes: a step of reacting cyanuric chloride with 2,6-diaminopyridine; and a step of mixing the reaction product with a metal salt of a counterion (X) of a secondary battery.

In an exemplary embodiment of the present disclosure, the counterion is one or more selected from a group consisting of Cl, BF₄, PF₆ and Tf₂N.

The present disclosure also provides a method for preparing an ionic organic framework for an all-solid secondary battery, which includes: a step of reacting cyanuric chloride with imidazole; and a step of mixing the reaction product with a metal salt of a counterion (X) of a secondary battery, wherein the counterion is one or more selected from a group consisting of Cl, BF₄, PF₆ and Tf₂N.

### Advantageous Effects

The structure and characteristics of a solid electrolyte, which is a core material of an all-solid lithium-ion secondary battery, are controlled effectively using an ionic organic framework. The solid electrolyte has excellent lithium-ion conductivity and stability.

### BRIEF DESCRIPTION OF THE DRAWINGS

FIG. 1 schematically shows a process of preparing ionic organic frameworks (ICOF-Xs) using different precursors.

FIG. 2 shows the FT-IR spectra of ICOFs depending on counteranions.

FIG. 3 shows the TEM images of a) ICOF-Cl-1, b) ICOF-BF₄-1, c) ICOF-PF₆-1, d) ICOF-Tf₂N-1 and e) ICOF-Cl-2.

FIG. 4 shows a) the powder XRD patterns (inset: magnified patterns from 15° to) 35° of ICOF-1s depending on counteranions and b) the powder XRD pattern and ionic framework structural simulation image of ICOF-2.

FIG. 5 shows a) the schematic diagram of a lithium coin cell using a solid electrolyte and b) the electrochemical impedance spectroscopy of a solid electrolyte prepared from ICOF-PF₆-1.

### BEST MODE FOR CARRYING OUT THE INVENTION

Hereinafter, the specific exemplary embodiments of the present disclosure will be described referring to the attached drawings. However, they are only examples and the present disclosure is not limited by them.

In the description of the present disclosure, if it is determined that a detailed description of a related known technology unnecessarily obscures the subject matter of the present disclosure, the detailed description will be omitted. Further, the terms used herein are defined in consideration of functions in the present disclosure, and may vary according to the intentions, customs, etc. of users or operators. Therefore, the definition should be made based on the contents throughout the disclosure. In addition, the scope of the present disclosure is defined by the appended claims and the exemplary embodiments described below are provided only to effectively explain the present disclosure to those having ordinary knowledge in the art to which the present disclosure belongs.

The present disclosure provides an electrolyte having crystallinity and porosity due to bonding between cyclic compounds. An organic framework according to the present disclosure contains a nitrogen-containing compound (e.g., pyridine or imidazole) and thus has an ammonium cation through protonation. As a result, a desired counteranion is easily bonded to the organic framework.

The structure wherein an ammonium cation is formed in the framework through protonation of the nitrogen-containing compound (e.g., pyridine or imidazole) and a counteranion is bonded to the cation is called an ionic organic framework (or ionic covalent organic framework (iCOF)).

In an exemplary embodiment of the present disclosure, the nitrogen which provides the cationic property in the ionic organic framework may be changed with phosphorus, etc. and the anion may also be changed freely, and this is encompassed in the scope of the present disclosure.

In addition, the anion contained in the organic framework may be controlled variously through post-treatment. Through this, the lithium-ion conductivity of a solid electrolyte based on the ionic organic framework may be adjusted and improved.

In an exemplary embodiment of the present disclosure, the nitrogen-containing organic compound for providing cationic property may be diaminopyridine, imidazole, etc., although not being limited thereto. And, the anion may be Cl, BF₄, PF₆, Tf₂N, etc., although not being limited thereto.

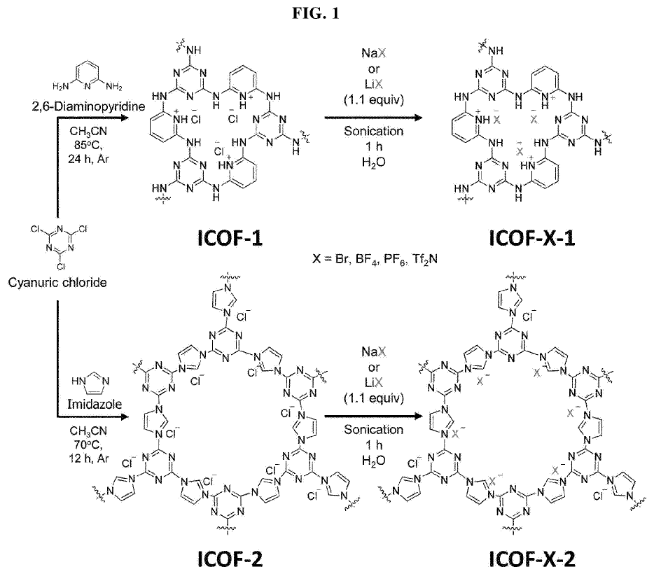

FIG. 1 schematically shows a process of preparing ionic organic frameworks (ICOF-Xs) using different precursors and organic frameworks prepared thereby.

Referring to FIG. 1, the organic framework according to an exemplary embodiment of the present disclosure has a framework structure wherein triazine and another hetero-organic compound (imidazole or pyridine) containing nitrogen as an aromatic cyclic element are connected with each other.

However, any aromatic cyclic material having three or more bonding sites in the unit compound may be used in the present disclosure. In the present disclosure, one large organic framework wherein an aromatic cyclic compound having at least three functional groups that can participate in condensation and an aromatic cyclic compound having two functional groups that can participate in condensation are connected in the unit aromatic cyclic compound through condensation is obtained.

In FIG. 1, the ICOF-X-1 organic framework has a structure wherein the triazine of cyanuric chloride is connected with the amine group of the pyridine of 2,6-diaminopyridine by reacting cyanuric chloride with 2,6-diaminopyridine, and the ICOF-X-2 organic framework has a structure wherein the triazine of cyanuric chloride is bonded to imidazole by reacting cyanuric chloride with imidazole.

The present disclosure may be changed variously and may have various exemplary embodiments. Hereinafter, the present disclosure will be described in detail through specific exemplary embodiments. However, it should be understood that the present disclosure is not limited by the specific exemplary embodiments but includes all changes, equivalents and substitutes encompassed in the scope of the present disclosure. In the following description, if it is determined that a detailed description of a related known technology unnecessarily obscures the subject matter of the present disclosure, the detailed description will be omitted.

### Example

Preparation of ICOF-1

Cyanuric chloride (0.2 M) and 2,6-diaminopyridine (0.3 M) were added to 40 mL of acetonitrile at 0° C. 1 hour later, the reaction mixture was stirred at 85° C. for 24 hours. The product was filtered using a 100-nm PVDF filter and washed three times with acetonitrile and acetone. ICOF-1 powder was obtained by drying the product overnight in vacuo.

Preparation of ICOF-2

Cyanuric chloride (0.2 M), imidazole (0.3 M) and N,N′-diisopropylethylamine (0.33 M) were added to 40 mL of acetonitrile at 0° C. 1 hour later, the reaction mixture was stirred at 70° C. for 12 hours. The product was filtered using a 100-nm PVDF filter and washed three times with acetonitrile and acetone. ICOF-2 powder was obtained by drying the product overnight in vacuo.

Preparation of ICOF-X

30 mL of H₂O and 100 mg of ICOF-1 or ICOF-2 and 1.1 equivalents of a metal salt of a counterion of a lithium battery (NaBF₄, NaPF₆ or LiTf₂N) were added to a 50-mL vial and sonicated for 1 hour in an ice bath using a probe tip. After centrifuging the reaction mixture at 10,000 rpm for 1 hour, the product was washed three times with H₂O and then freeze-dried overnight.

Preparation of Solid Electrolyte

150 mg of the ICOF-x was mixed with 150 mg of a PTFE solution of two drops of LiTf₂N (10 wt %, NMP). Electrolyte pellets were obtained by compressing the product at 2 tons for 10 minutes. The pellets were dried at 60° C. to remove the residual solvent.

### Test Example

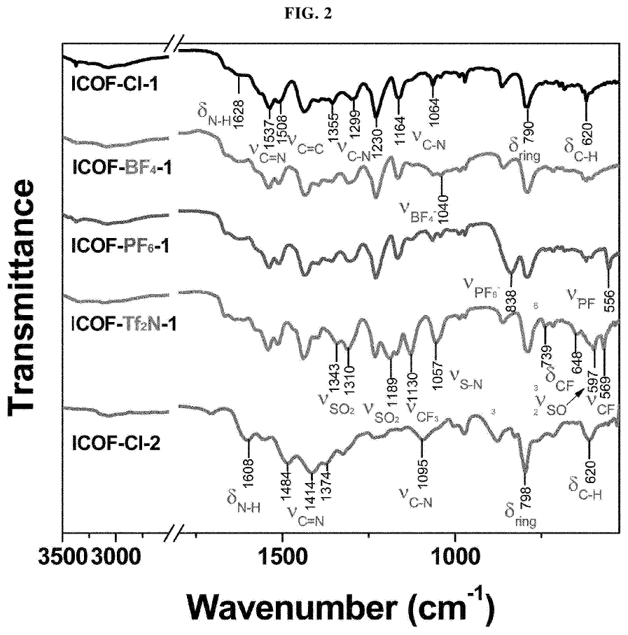

FIG. 2 shows the FT-IR spectra of the ICOFs depending on counteranions. The structure of ICOF-1 and ICOF-X-1 was analyzed by Fourier-transform infrared spectroscopy (FT-IR) as shown in FIG. 2.

Referring to FIG. 2, it can be seen that characteristic peaks corresponding to the anions X appear as the anion the ICOF-X-1 is varied. It means that the anionic property of the ionic organic framework according to the present disclosure can be changed by varying the anion and the conductivity, etc. of the electrolyte can be controlled freely.

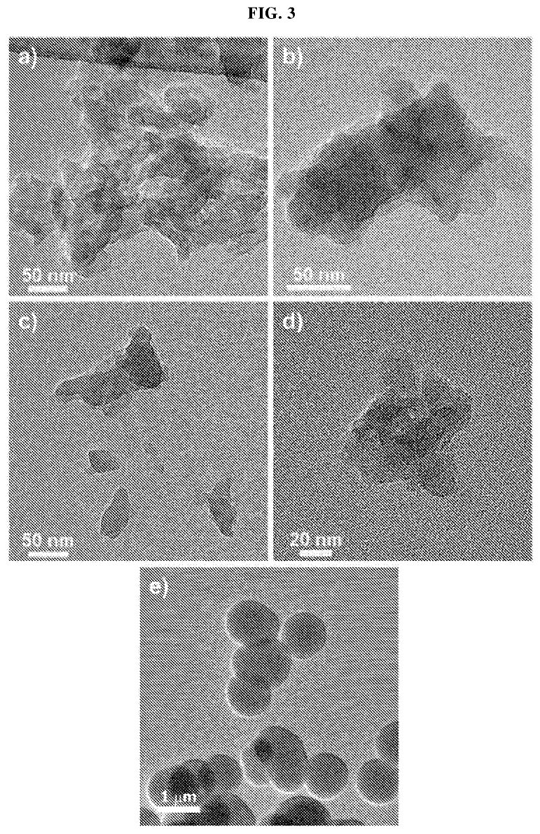

FIG. 3 shows the TEM images of a) ICOF-Cl-1, b) ICOF-BF₄-1, c) ICOF-PF₆-1, d) ICOF-Tf₂N-1 and e) ICOF-Cl-2.

Referring to FIG. 3, it can be seen that anion-bonded powders for an electrolyte can be prepared effectively using various metal salts of anions according to the method of the present disclosure.

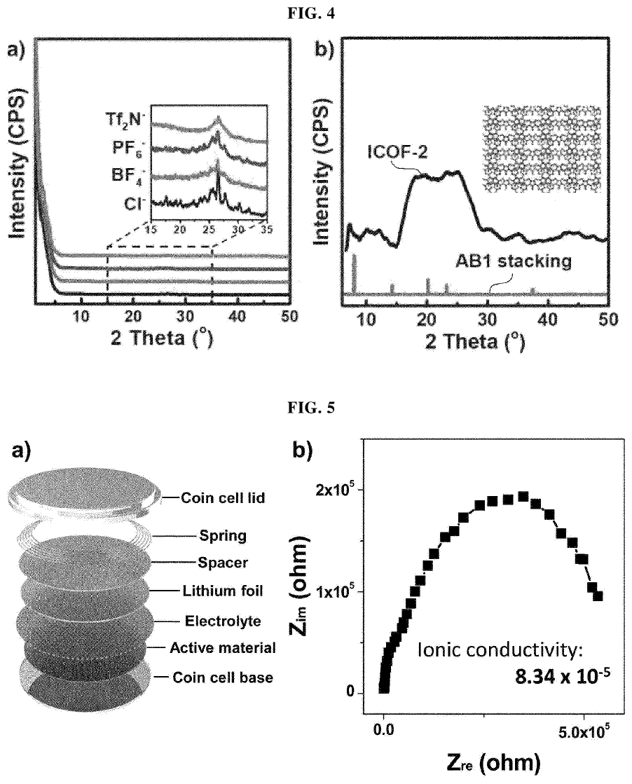

FIG. 4 shows a) the powder XRD patterns (inset: magnified patterns from 15° to 35°) of ICOF-1s depending on counteranions and b) the powder XRD pattern and ionic framework structural simulation image of ICOF-2.

In FIG. 4, a smaller 2 theta value means a larger framework structure. The strong peak at <5° verifies that the framework structure of the present disclosure is an “organic framework”, not a polymer type.

FIG. 5 shows a) the schematic diagram of a lithium coin cell using a solid electrolyte and b) a result of measuring electrical conductivity.

Referring to FIG. 5, the all-solid battery using the ionic organic framework-based electrolyte prepared according to an exemplary embodiment of the present disclosure exhibits higher conductivity as compared to the existing electrolyte.

While the present disclosure has been described in detail with respect to the specific exemplary embodiments, it will be obvious to those having ordinary knowledge in the art will that they are merely specific exemplary embodiments, and the scope of the present disclosure is not limited by them. It is to be noted that the substantial scope of the present disclosure is defined by the appended claims and their equivalents.

1. An ionic organic framework for an all-solid secondary battery, wherein the organic framework comprises a compound capable of forming a cation through protonation in the framework, a counteranion for a secondary battery is bonded to the cation, and the organic framework has crystallinity.

2. The ionic organic framework for an all-solid secondary battery of claim 1, wherein the organic framework is formed from condensation between an aromatic compound having at least three functional groups that can participate in condensation and another aromatic compound having two or more functional groups that can participate in condensation.

3. The ionic organic framework for an all-solid secondary battery of claim 2, wherein the aromatic compound is triazine and the another aromatic compound is at least one of pyridine or imidazole.

4. The ionic organic framework for an all-solid secondary battery of claim 1, wherein the ionic organic framework for an all-solid secondary battery has a structure of Chemical Formula (1) or Chemical Formula (2): 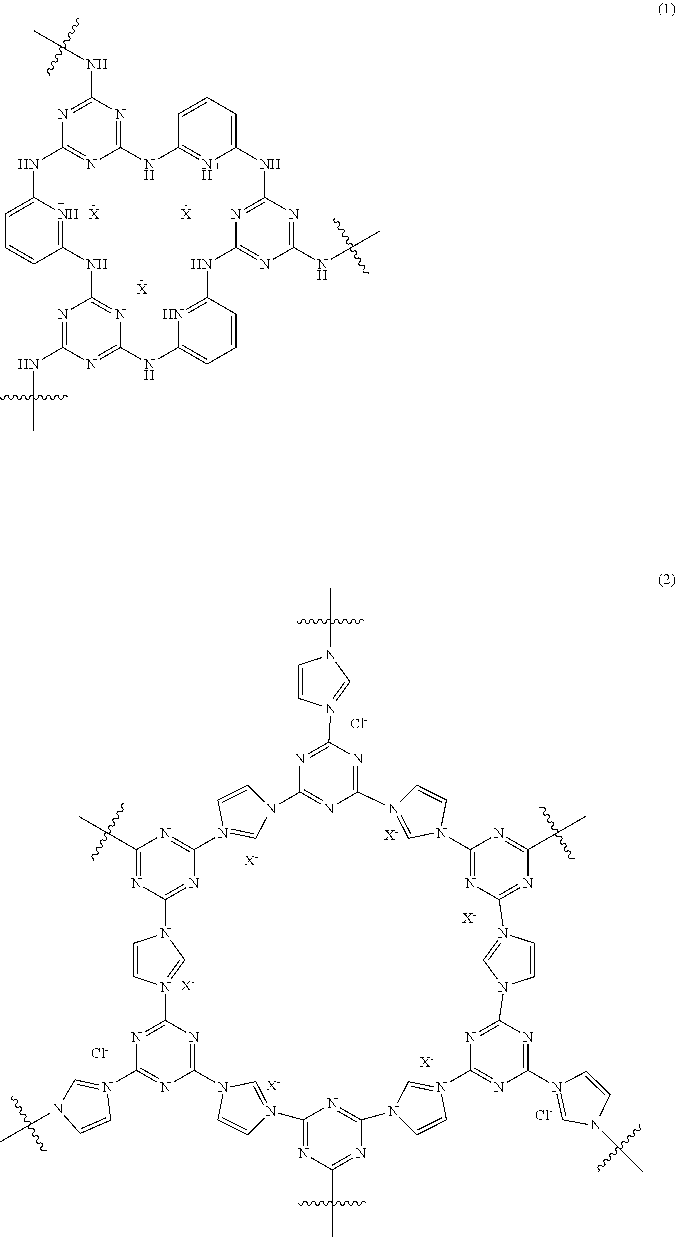
wherein X is a counterion of a lithium battery.

5. The ionic organic framework for an all-solid secondary battery of claim 1, wherein the organic framework is for an electrolyte of an all-solid secondary battery.

6. The ionic organic framework for an all-solid secondary battery of claim 4, wherein the organic framework of Formula (1) is obtained by reacting cyanuric chloride with 2,6-diaminopyridine and has a structure wherein the triazine of the cyanuric chloride is connected to the pyridine of the 2,6-diaminopyridine by an amine group.

7. The ionic organic framework for an all-solid secondary battery of claim 4, wherein the organic framework of Formula (2) is obtained by reacting cyanuric chloride with imidazole and has a structure wherein the triazine of the cyanuric chloride is bonded to the imidazole.

8. An electrolyte comprising the ionic organic framework for an all-solid secondary battery of claim 1.

9. An all-solid secondary battery comprising the electrolyte of claim 8.

10-13. (canceled)

14. The ionic organic framework for an all-solid secondary battery of claim 1, wherein the counterion is one or more selected from the group consisting of Cl, BF₄, PF₆, and Tf₂N.
